(19) United States
(12) Patent Application Publication
(10) Pub. No.: US 2026/0144551 A1
(43) Pub. Date: May 28, 2026

(54) Integrated Tourniquet Garment Device

(71) Applicant: Jessica Aguilar, Roxboro, NC (US)

(72) Inventor: Jessica Aguilar, Roxboro, NC (US)

(21) Appl. No.: 19/321,778

(22) Filed: Sep. 8, 2025

Related U.S. Application Data
(60) Provisional application No. 63/724,645, filed on Nov. 25, 2024.

(51) Int. Cl. A61B 17/132; A41B 1/08; A41D 1/06; A41D 7/00; B63C 11/02; B63C 11/04

(52) U.S. Cl. CPC A61B 17/1322; A41B 1/08; A41D 1/06; A41D 7/00; B63C 11/04; B63C 2011/023; B63C 2011/046

(57) ABSTRACT

An integrated tourniquet garment device is provided. The device is comprised of one or more garments with embedded tourniquet mechanisms disposed at critical anatomical pressure points along the upper and lower limbs. The garments comprise either two-piece or one-piece configurations, including but not limited to tactical uniforms or wetsuit structures. The tourniquet mechanisms comprise a tensioning assembly such as a nylon web band, a double-D clasp, and an externally accessible pull tab configured for circumferential compression of limb segments. The mechanisms are inlaid within seam structures or enclosed pockets at locations including but not limited to the elbows, armpits, upper thighs, and knees, with additional optional placement at the forearms, calves, or ankles. The pull tab allows manual actuation, with the tourniquet tension maintained via the double-D clasp in a locked configuration.

### CROSS-REFERENCE TO RELATED APPLICATION

The present application claims priority to, and the benefit of, U.S. Provisional Application No. 63/724,645, which was filed on Nov. 25, 2024, and is incorporated herein by reference in its entirety.

### FIELD OF THE INVENTION

The present invention relates generally to the field of tourniquets. More specifically, the present invention relates to one or more garments incorporating embedded tourniquet mechanisms positioned at key anatomical pressure points on the upper and lower limbs to enable circumferential compression for hemorrhage control. Accordingly, the present disclosure makes specific reference thereto. Nonetheless, it is to be appreciated that aspects of the present invention are also equally applicable to other like applications, devices, and methods of manufacture.

### BACKGROUND

Traumatic injuries to the extremities present a critical risk of severe blood loss, which can be fatal if not addressed immediately. In many fields or emergency scenarios, especially those involving combat, natural disasters, or remote environments, access to advanced medical care is delayed or unavailable. In such contexts, hemorrhage control becomes the highest priority for sustaining life. The application of a tourniquet is a well-established emergency intervention used to mitigate this threat by restricting blood flow to the affected limb. However, tourniquet application, particularly self-application or application under duress, can be difficult even for trained professionals. The process typically requires separate equipment, time, and physical dexterity, which may not be feasible in high-stress environments. Furthermore, improper application can result in either insufficient pressure to stop bleeding or excessive pressure leading to tissue damage. The ability to intermittently release and re-tighten a tourniquet to preserve limb viability introduces additional complexity that cannot always be managed in the field. There also exists the problem of access to reliable and durable tourniquet materials, which may not be readily available in every emergency. In recognition of these challenges, a solution is needed that integrates hemorrhage control mechanisms directly into clothing to improve accessibility, ease of use, and efficiency in high-risk environments.

Therefore, there exists a long-felt need in the art for an integrated tourniquet garment device that enables rapid, self-administered hemorrhage control without requiring separate equipment. There also exists a long-felt need in the art for an integrated tourniquet garment device that permits intermittent loosening and re-tightening to balance blood flow and limb preservation. Moreover, there exists a long-felt need in the art for an integrated tourniquet garment device that maintains operational utility, mobility, and durability in varied environmental and tactical conditions.

The subject matter disclosed and claimed herein, in one embodiment thereof, comprises an integrated tourniquet garment device. The device is comprised of one or more garments with embedded tourniquet mechanisms disposed at critical anatomical pressure points along the upper and lower limbs. The garments comprise either two-piece or one-piece configurations, including but not limited to tactical uniforms or wetsuit structures. The tourniquet mechanisms comprise a tensioning assembly such as a nylon web band, a double-D clasp, and an externally accessible pull tab configured for circumferential compression of limb segments. The mechanisms are inlaid within seam structures or enclosed pockets at locations including but not limited to the elbows, armpits, upper thighs, and knees, with additional optional placement at the forearms, calves, or ankles. The pull tab allows manual actuation, with the tourniquet tension maintained via the double-D clasp in a locked configuration. The device further permits controlled pressure release and re-tightening at intervals to balance hemorrhage control with limb perfusion.

In this manner, the integrated tourniquet garment device of the present invention accomplishes all the forgoing objectives and integrates life-saving hemorrhage control systems directly into the structure of a garment, thereby eliminating the need for external tourniquet equipment. The device allows for rapid self-application or assisted application in emergency scenarios while preserving wearer mobility and operational readiness. The embedded mechanisms further permit controlled compression and decompression cycles, thereby supporting both immediate trauma response and extended limb preservation under variable field conditions. As such, the integrated tourniquet garment device effectively addresses the problems associated with traditional tourniquet use in tactical, remote, or high-risk environments.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an integrated tourniquet garment device. The device is comprised of garments with integrated tourniquet systems to enable rapid on-body hemorrhage control in emergency, tactical, or remote environments. The integrated systems allow localized pressure application to limbs or joints without requiring separate tourniquet equipment. The device is designed to preserve mobility, concealment, and operational function while incorporating life-saving mechanisms into the garment structure.

The device may be configured as a two-piece tactical uniform, a unitary wetsuit, or alternative clothing configurations. In one embodiment, the two-piece configuration is comprised of a long sleeve shirt component and a pant component. Each garment is further comprised of at least one integrated tourniquet mechanism positioned at critical anatomical pressure points on the garment. In one embodiment, the tourniquet mechanisms are embedded through seam structures at up to eight bilateral locations including elbows, armpits, upper thighs, and knees. Additional placements may include forearms, calves, or ankles. These positions are selected based on proximity to major arteries and joint creases to optimize pressure application while preserving mobility.

In one embodiment of the long sleeve shirt, tourniquet mechanisms are located at the elbow and underarm regions. For the pant component, placements include the top of the thigh and the top of the knee. Each mechanism may be embedded directly into garment inseams or integrated into internal pockets within the garment lining.

Each tourniquet mechanism may be comprised of a tensioning assembly including a nylon web band, a double-D clasp, and a pull tab. The nylon web band is fixed at a proximal end to the double-D clasp and pre-threaded through it, terminating in a pull tab that extends externally through an opening in the garment for manual operation. Activation of the tourniquet mechanism is performed by applying tensile force to the pull tab, tightening the nylon band around the targeted limb segment. The pull tab may be doubled back through the clasp to lock the band in place, maintaining pressure and preventing loosening. The configuration allows controlled loosening and re-tightening intervals for limb perfusion management. The mechanisms may be enclosed in sealed pockets, with only the clasp and pull tab accessible externally to reduce snag risk and environmental exposure.

In an alternate embodiment, the garment may be a one-piece wetsuit configuration. This version incorporates equivalent tourniquet placements and is comprised of stretchable, water-resistant materials such as neoprene or coated spandex composites to ensure a conformal fit. The wetsuit may include an elastic stirrup at the distal end of each leg to secure the user's foot and prevent axial displacement during movement. The wetsuit embodiment may also comprise protective panels located on the arm and leg portions, distal to the tourniquet mechanisms. These panels provide shielding against impact, abrasion, or puncture.

Accordingly, the integrated tourniquet garment device of the present invention is particularly advantageous as it integrates life-saving hemorrhage control systems directly into the structure of a garment, thereby eliminating the need for external tourniquet equipment. The device allows for rapid self-application or assisted application in emergency scenarios while preserving wearer mobility and operational readiness. The embedded mechanisms further permit controlled compression and decompression cycles, thereby supporting both immediate trauma response and extended limb preservation under variable field conditions. As such, the integrated tourniquet garment device effectively overcomes the problems and limitations associated with traditional tourniquet use in tactical, remote, or high-risk environments.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front view of one potential embodiment of an integrated tourniquet garment device of the present invention wherein the garment is a shirt in accordance with the disclosed architecture;

FIG. 2 illustrates a front view of one potential embodiment of an integrated tourniquet garment device of the present invention wherein the garment is a pant in accordance with the disclosed architecture; and

FIG. 3 illustrates a front view of one potential embodiment of an integrated tourniquet garment device of the present invention wherein the garment is a wetsuit in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an integrated tourniquet garment device that enables rapid, self-administered hemorrhage control without requiring separate equipment. There also exists a long-felt need in the art for an integrated tourniquet garment device that permits intermittent loosening and re-tightening to balance blood flow and limb preservation. Moreover, there exists a long-felt need in the art for an integrated tourniquet garment device that maintains operational utility, mobility, and durability in varied environmental and tactical conditions.

The present invention, in one exemplary embodiment, is comprised of an integrated tourniquet garment device. The device is comprised of a garment incorporating integrated tourniquet systems, enabling rapid on-body hemorrhage control suitable for emergency, tactical, or remote applications. These systems facilitate direct application of localized pressure to limbs or joints without relying on separate tourniquet equipment. The garment design maintains wearer mobility, concealment, and operational capability while embedding life-saving features into the structural fabric of the clothing.

The device may be implemented as a two-piece tactical uniform, a unitary wetsuit, or other garment configurations. In one embodiment, the two-piece uniform includes a long-sleeve shirt component and a pant component. Each garment is further equipped with at least one integrated tourniquet mechanism located at key anatomical pressure points. In one configuration, tourniquet mechanisms are embedded within seam structures at up to eight bilateral sites, including elbows, armpits, upper thighs, and knees. Additional placements may include forearms, calves, or ankles. These anatomical zones are selected based on proximity to major arteries and joint flexion areas, ensuring optimal pressure application while preserving mobility.

In one example of the long sleeve shirt configuration, the tourniquet mechanisms are positioned at the elbow and underarm regions. On the pant component, mechanisms are located at the upper thigh and knee areas. Each tourniquet mechanism may be embedded directly into garment inseams or housed within internal pockets in the garment lining.

Each tourniquet mechanism includes a tensioning assembly comprising a nylon web band, a double-D clasp, and a pull tab. The nylon web band is anchored at its proximal end to the double-D clasp, threaded through the clasp, and terminates at a pull tab that exits through an external garment opening for manual engagement. Activation is achieved by pulling the tab to apply tensile force, thereby tightening the band around the limb. The pull tab may then be looped back through the clasp to lock the band in a secured position, ensuring consistent pressure and preventing unintentional loosening. The device allows for controlled loosening and re-tightening cycles to support intermittent limb perfusion. The mechanism may be enclosed in a sealed pocket with only the clasp and pull tab accessible externally, minimizing environmental exposure and snag risks.

In an alternate embodiment, the device may take the form of a one-piece wetsuit. This configuration retains similar tourniquet placements and is fabricated from stretchable, water-resistant materials such as neoprene or coated spandex to provide a conformal, hydrostatic fit. Each leg portion may include an integrated elastic stirrup at its distal end to secure the foot and prevent axial garment displacement during movement. Additionally, the wetsuit may incorporate protective panels on the arm and leg sections, positioned distal to the tourniquet mechanisms, to protect extremities from impact, abrasion, or puncture.

As a result, the device provides embedded hemorrhage control systems directly into a garment structure, thereby eliminating reliance on external tourniquet tools. The device further enables rapid self-application or assisted application in emergency situations while preserving mobility and readiness. The internal mechanisms support adjustable compression cycles, facilitating both immediate trauma response and extended limb preservation under varied operational conditions. Therefore, the device addresses the challenges and limitations associated with traditional tourniquet systems in high-risk, tactical, or remote environments.

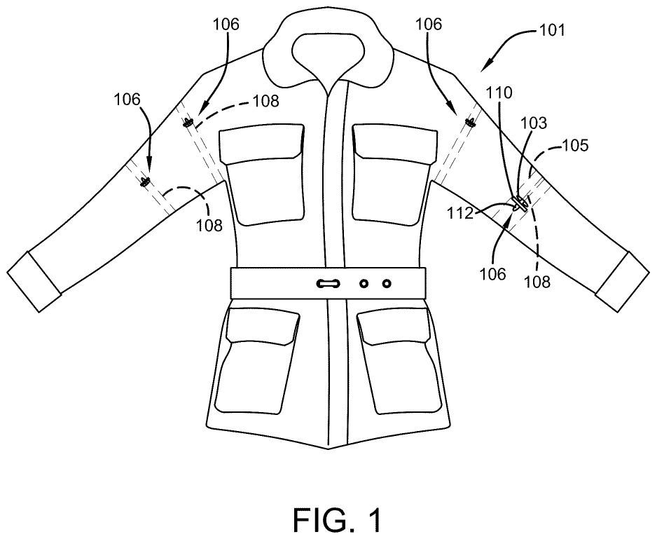

Referring initially to the drawings, FIG. 1 illustrates a front view of one potential embodiment of an integrated tourniquet garment device 100 of the present invention wherein the garment 101 is a shirt 102 in accordance with the disclosed architecture. The device 100 is comprised of garments 101 with integrated tourniquet systems 106 to address the need for rapid, on-body hemorrhage control in emergency, tactical, or remote environments. The device 100 enables wearers or first responders to apply localized pressure to limbs or joints without requiring separate tourniquet equipment to be applied to the wounded individual. The device 100 is further designed to maintain mobility, concealment, and operational utility while embedding critical life-saving features directly into the structural configuration of the garment.

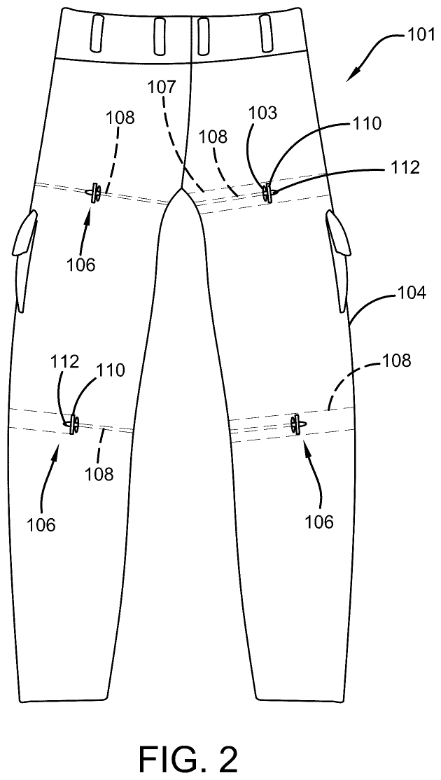

In various embodiments, the device 100 may be a two-piece tactical uniform, a unitary wetsuit, or any other clothing or uniform configuration. In one embodiment, the two-piece configuration may be comprised of garments 101 such as a long sleeve shirt component 102 and a pant component 104, as seen in FIG. 2. Additional variations of the shirt component 102 and pant component 104 may include but are not limited to any combination of short sleeve shirts, sleeveless vests, shorts, or capri-length pants to accommodate different climates, mission parameters, and operational roles. In certain configurations, garments 101 may be designed for layering compatibility with base or outer garments, allowing the tourniquet functionality to remain accessible regardless of the thermal or protective layers worn above or below.

The device 100 is comprised of at least one integrated tourniquet mechanism 106 disposed at key anatomical pressure points on the garments 101. In one embodiment, the tourniquet mechanisms 106 are inlayed within the garments 101 via seam structures 107 at eight critical locations including but not limited to any combination of bilateral locations such as elbows, armpits, upper thighs, and knees, as seen in FIG. 1 and FIG. 2. Additional embodiments may position tourniquet mechanisms 106 at the forearms, calves, or ankles to address lower extremity hemorrhages or peripheral trauma in compact body zones. These locations correspond to high-risk zones for hemorrhage control and may be selected based on proximity to major arteries such as the brachial, femoral, or popliteal arteries. The placement of the tourniquet mechanisms 106 may further correspond to natural creases or flexion zones of the limbs to minimize interference with range of motion and maximize pressure distribution.

More specifically, in a long sleeve shirt 102 configuration, the tourniquet mechanisms 106 may be located at the arm crease adjacent to the elbow and at the shoulder crease adjacent to the underarm. On the pant configuration 104, the tourniquet mechanisms 106 may be located at the top of the thigh and at the top of the knee. In each instance, the entirety and/or a portion of the tourniquet mechanisms 106 may be inlayed directly into the inseam of the garment 101 or into a dedicated pocket 105 in the inner lining of the garment 101.

Each tourniquet mechanism 106 may be comprised of a tensioning assembly including but not limited to a nylon web band 108, a double-D clasp 110, and a pull tab 112. The nylon web band 108 may be fixed at a proximal end to a central portion of the double-D clasp 110. The distal end of the nylon web band 108 may be pre-threaded through the double-D clasp 110 and terminate in the pull tab 112, which protrudes externally through an opening 103 of the garment 101 for manual actuation.

The integrated tourniquet mechanisms 106 may be various tourniquet types such as but not limited to any combination of nylon web and clasp assemblies, ratcheting strap systems, inflatable bladder systems, twist-rod (windlass) configurations, and cable-pulley tensioning assemblies. Each type may offer distinct advantages in terms of adjustability, compression force, actuation time, and durability. These variants may be implemented individually or in combination in various embodiments of the device 100 to provide adaptable hemorrhage control across different operational scenarios.

To activate the tourniquet mechanism 106, the user or a first responder may apply tensile force to the pull tab 112, thereby tightening the nylon web band 108 circumferentially around the targeted limb segment of the user. Once tightened, the pull tab 112 may be doubled back through the double-D clasp 110 in a locking configuration, securing the nylon web band 108 in the tightened or active position. This configuration may prevent loosening due to movement and maintain consistent pressure on the compressed area. Additionally, the configuration may permit incremental loosening and retightening cycles at defined intervals, such as every 15 minutes, to balance hemorrhage control with limb perfusion. Each band 108 may be fully enclosed within a sealed pocket 105 of the garment 101, with only the pull tab 112 and double-D clasp 110 accessible from the exterior. This configuration reduces snag risk, prevents environmental contamination, and maintains the external appearance of the device 100 while preserving immediate access to the hemorrhage control function.

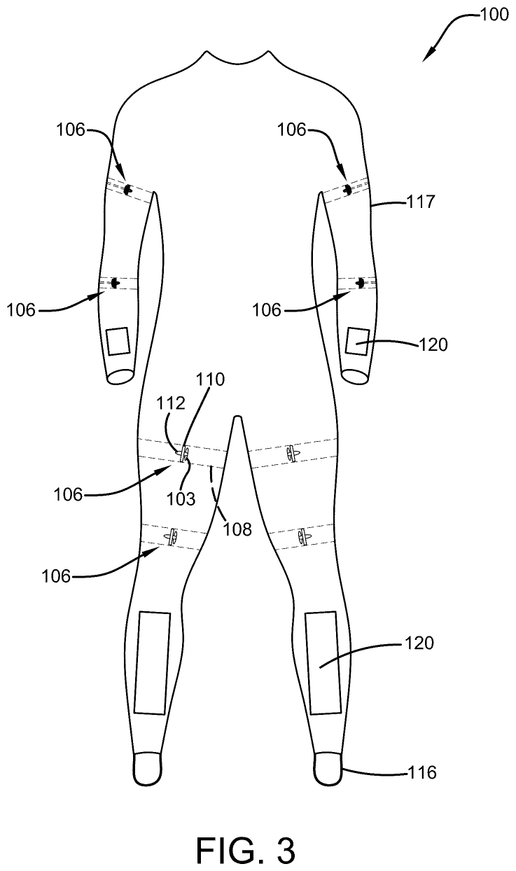

In an alternate embodiment, the garment 101 may be comprised of a unitary one-piece wetsuit configuration, as seen in FIG. 3. The wetsuit configuration may similarly integrate tourniquet mechanisms 106 at equivalent anatomical positions to those previously described. In this configuration, the garment 101 may be formed from stretchable, water-resistant materials such as but not limited to neoprene or coated spandex composites to ensure conformal fit and hydrostatic performance. In the wetsuit embodiment, the device 100 may further comprise at least one elastic stirrup 116 integrally formed at a distal end of each leg 118. Each stirrup 116 may be configured to retain a user's foot and prevent axial displacement of the device 100 during movement.

Additionally, the wetsuit embodiment may further comprise protective panels 120 disposed on each arm portion 117 and each leg portion 118. Each protective panel 120 may be positioned distal to the associated tourniquet mechanism 106 to shield vulnerable extremity zones from impact, abrasion, or puncture hazards. The panels 120 may be comprised of abrasion-resistant or impact-dissipating materials such as but not limited to neoprene, ballistic nylon, thermoplastic urethane, aramid composites, or foam-laminated structures.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “integrated tourniquet garment device” and “device” are interchangeable and refer to the integrated tourniquet garment device 100 of the present invention.

Notwithstanding the foregoing, the integrated tourniquet garment device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the integrated tourniquet garment device 100 as shown in the FIGS. are for illustrative purposes only, and that many other sizes and shapes of the integrated tourniquet garment device 100 are well within the scope of the present disclosure. Although the dimensions of the integrated tourniquet garment device 100 are important design parameters for user convenience, the integrated tourniquet garment device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An integrated tourniquet garment device comprising:
a garment comprising a tourniquet mechanism integrated into the garment;
wherein the tourniquet mechanism comprises a band, a clasp, and a pull tab; and
wherein the pull tab is attached to the band and extends through an opening of the garment.

2. The integrated tourniquet garment device of claim 1, wherein the garment is comprised of a shirt.

3. The integrated tourniquet garment device of claim 1, wherein the garment is comprised of a pant.

4. The integrated tourniquet garment device of claim 2, wherein the tourniquet mechanism is positioned at an elbow or an armpit of the shirt.

5. The integrated tourniquet garment device of claim 3, wherein the tourniquet mechanism is positioned at a knee or a thigh of the pant.

6. The integrated tourniquet garment device of claim 1, wherein the clasp is comprised of a double-D clasp.

7. The integrated tourniquet garment device of claim 3, wherein the tourniquet mechanism is positioned in a pocket of the garment.

8. An integrated tourniquet garment device comprising:
a garment comprising a tourniquet mechanism positioned within an inner lining of the garment;
wherein the tourniquet mechanism comprises a band, a clasp, and a pull tab; and
further wherein the pull tab is attached to the band and extends through an opening of the garment.

9. The integrated tourniquet garment device of claim 8, wherein the garment is comprised of a shirt.

10. The integrated tourniquet garment device of claim 8, wherein the garment is comprised of a pant.

11. The integrated tourniquet garment device of claim 9, wherein the tourniquet mechanism is positioned at an elbow or an armpit of the shirt.

12. The integrated tourniquet garment device of claim 10, wherein the tourniquet mechanism is positioned at a knee or a thigh of the pant.

13. The integrated tourniquet garment device of claim 8, wherein the clasp is comprised of a double-D clasp.

14. An integrated tourniquet garment device comprising:
a wetsuit comprising a protective panel, a stirrup, and a tourniquet mechanism integrated into the wetsuit;
wherein the tourniquet mechanism comprises a band, a clasp, and a pull tab;
wherein the pull tab is attached to the band and extends through an opening of the wetsuit; and
a protective panel.

15. The integrated tourniquet garment device of claim 14, wherein the tourniquet mechanism is positioned at an elbow or an armpit of the wetsuit.

16. The integrated tourniquet garment device of claim 14, wherein the tourniquet mechanism is positioned at a knee or a thigh of the wetsuit.

17. The integrated tourniquet garment device of claim 14, wherein the protective panel is comprised of a neoprene, a ballistic nylon, a thermoplastic urethane, an aramid composite, or a foam-laminated structure.

18. The integrated tourniquet garment device of claim 14, wherein the stirrup is comprised of an elastic stirrup.

19. The integrated tourniquet garment device of claim 14, wherein the wetsuit is comprised of a neoprene or a coated spandex.

20. The integrated tourniquet garment device of claim 14, wherein the protective panel is positioned on an arm or a leg of the wetsuit.
